(19) United States
(12) Patent Application Publication
(10) Pub. No.: US 2013/0204203 A1
(43) Pub. Date: Aug. 8, 2013

(54) Drug Delivery Device

(71) Applicants: Lutz Mueller, Frankfurt am Main (DE); Claudia Matthias, Frankfurt am Main (DE); Michael Helmer, Frankfurt am Main (DE); Steffen Raab, Frankfurt am Main (DE); Axel Forstreutter, Frankfurt am Main (DE)

(72) Inventors: Lutz Mueller, Frankfurt am Main (DE); Claudia Matthias, Frankfurt am Main (DE); Michael Helmer, Frankfurt am Main (DE); Steffen Raab, Frankfurt am Main (DE); Axel Forstreutter, Frankfurt am Main (DE)

(73) Assignee: SANOFI-AVENTIS DEUTSCHLAND GMBH, Frankfurt am Main (DE)

(21) Appl. No.: 13/576,671

(22) PCT Filed: Mar. 16, 2011
(86) PCT No.: PCT/EP2011/053987

§ 371 Date: Jan. 17, 2013

(30) Foreign Application Priority Data
Mar. 16, 2010 (EP) 10156586.9

(51) Int. Cl. A61M 5/315

(52) U.S. Cl. CPC A61M 5/31548
USPC 604/208; 29/428

(57) ABSTRACT

The present invention relates to a drug delivery device for dispensing of a dose of a medicinal product, comprising:
a first housing component (12, 52; 62) adapted to receive a cartridge (16) comprising the medicinal product and further comprising a piston (18) slidably arranged therein in an axial direction,
a second housing component (14; 54; 64) adapted to receive a dose dispensing mechanism having a piston rod (20) to be operably engaged with the cartridge's piston (18),
wherein the first and the second housing components (12, 14; 52, 54; 62, 64) are displaceably arranged in axial direction relative to each other for reducing an axial distance (34) between the piston (18) and the piston rod (20) to a pre-defined degree, and
an interlock means (30) adapted to interact with at least one of first and second housing components (12, 14; 52, 54; 62, 64) for mutually locking into position first and second housing components (12, 14; 52, 54; 62, 64).

This invention relates to a drive mechanism for a drug delivery device that allows a user to select single or multiple doses of an injectable medicinal product and to dispense the set dosage of the product and to apply said product to a patient, preferably by injection. In particular, the present invention relates to such devices, which are handled by the patients themselves.

Drug delivery devices allowing for multiple dosing of a required dosage of a liquid medicinal product, such as liquid drugs, and further providing administration of the liquid to a patient, are as such well-known in the art. Generally, such devices have substantially the same purpose as that of an ordinary syringe.

Drug delivery devices of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reducible, the device should be inexpensive to manufacture and easy to dispose. In order to meet these requirements, the number of parts and steps required to assemble the device and an overall number of material types the device is made from have to be kept to a minimum.

Typically, the medicinal product to be administered is provided in a cartridge that has a moveable piston or bung mechanically interacting with a piston rod of a drive mechanism of the drug delivery device. By applying thrust to the piston in distal direction, a certain amount of the medicinal fluid is expelled from the cartridge.

Due to inevitable manufacturing tolerances there may for instance persist axial clearance between a cartridge's piston and the piston rod. Typically, prior to a primary use of the device and/or a primary use after a cartridge replacement, the device has to be prepared for an initial injection by a so-called set-up procedure. Hence, prior to injection it should be ensured, that the piston rod and the piston mutually abut or that an inevitable gap between piston rod and piston is reduced to a minimum. If piston and piston rod get in direct contact with each other, a distally directed displacement of the piston rod during an injection procedure can be directly transferred to a respective displacement of the piston. Typically, such a set-up procedure has to be driven manually by the user in order to ensure that already with an initial dose setting and a subsequent dose dispensing step, a predefined amount of the medicinal product is dispensed in an accurate way.

Since a self-administering user might be physically infirm, it is desirable to simplify or even to eliminate the need for such a user-conductible set-up procedure.

Document DE 195 19 147 A1 describes an injection apparatus comprising a housing and a container for an injection fluid, wherein the container is slidably disposed between a distal and proximal end position relative to the housing. Preferably, the container is retained by a holder, which is adjustable in length. The holder comprises a proximal and a distal section being for instance mutually coupled by means of a micro-detent mechanism. By way of exerting an axially directed force, the total axial length of the holder may be reduced, such as to eliminate axial clearance between the container and a plunger of a drive mechanism.

Even though such known apparatus is generally applicable to eliminate axial clearance, the length-adjusting mechanism making use of micro-detents comes along with some drawbacks.

The micro-detent mechanism inherently provides a certain mechanical resistance against axial displacement of distal end proximal sections of the holder. On the one hand and in particular for axial clearance elimination, mechanical resistance provided by the micro-detent mechanism should be rather low in order to allow for a smooth adjustment. On the other hand, after adjustment of the holder, the micro-detent mechanism has to provide a mechanical resistance being appropriate to withstand axial forces arising during dose selecting and dose dispensing under typical operation conditions.

If the resistivity provided by the micro-detent mechanism is too small, e.g. during a dose dispensing action, the adjustment of the holder will eventually become subject to unintentional modifications. This may in turn lead to respective modifications of the amount of liquid to be dispensed and may have severe consequences for the health of the patient. Otherwise, if the mechanical resistance provided by the micro-detent mechanism is too large, a clearance-eliminating adjustment of the holder will be hindered. Moreover, since larger axial forces have to be applied, a haptic feedback, obtainable when piston rod and piston mutually abut, may be difficult to detect. At the end there may arise a certain risk, that in the course of clearance-elimination, a certain amount of injection fluid is spilled or unintentionally expelled.

It is therefore an object of the present invention, to provide a drug delivery device featuring improved and facilitated clearance as well as manufacturing tolerance elimination. It is a further object of the invention, to redundantize a set-up procedure usually to be conducted by the end user. The invention further focuses on improvements related to patient safety and intends to simplify construction and handling of the device. It is another object of the invention to provide a method of eliminating clearance between the piston and piston rod and to facilitate a set-up procedure prior to an initial use of the drug delivery device.

In a first aspect, the invention provides a drug delivery device for dispensing of a dose of a medicinal product, typically a medicinal fluid, such as a fluid drug.

The drug delivery device comprises a first housing component, which is adapted to receive a cartridge. The cartridge comprises the medicinal product to be dispensed and further comprises a piston being slidably arranged therein in an axial direction. For dispensing of a predefined dose of the medicinal product, the drug delivery device further has a dose dispensing mechanism arranged in a second housing component. The dose dispensing mechanism has a piston rod to be operably engaged with the cartridge's piston. With a distal outlet, the cartridge may be engaged with a needle, a cannula, an infusion tube or similar delivery devices in a fluid-transferring way. The cartridge itself may be designed as replaceable or disposable ampoule, carpule or syringe. Its piston is displaceable at least in distal direction for purging or expelling a pre-defined dose of medicinal product from the cartridge.

The first housing component and the second housing component, are displaceably arranged in axial direction relative to each other. By way of the at least two mutually axially displaceable housing components, axial clearance or an axial distance between the piston and the piston rod can be eliminated or at least reduced to a pre-defined degree. Further, there is provided an interlock means, which is adapted to interact with at least one of first and second housing components. By way of the interlock means, first and second housing components can be locked into position, preferably in such a position, in which axial clearance between piston and piston rod is entirely eliminated and in which piston and piston rod mutually abut and get in contact with each other.

By making use of particularly designed interlock means, axial clearance- or backslash elimination can be optimized and simplified. The mutual displacement of first and second housing components can be designed and adapted to provide a smooth-running axial relative displacement featuring a minimal mechanical resistance. Furthermore, the interlock means itself provides an effective means to block and to immobilize first and second housing components after the clearance-elimination procedure has been conducted. By activating the interlock means, mechanical resistance for relative and axial movement of first and second housing components can be increased to a predefined level, which is preferably beyond maximal axially directed forces arising during normal operation of the device.

The present invention therefore provides a structural and/or constructive separation of a relative displacement of housing components on the one hand side and their mechanical coupling, hence mutual interlocking in a clearance-free configuration, on the other hand side. In this way, a clearance-eliminating movement of housing components can be conducted irrespective of their mutual fixing or blocking. In the opposite sense, also the interlock means for fixing first and second housing components relative to each other can be designed and adapted irrespective of the displaceable arrangement of first and second housing components.

Therefore, the invention provides an approach to selectively optimize a clearance-eliminating relative displacement of first and second housing components and a respective fixing of said components in separate and uncorrelated ways.

The mutually displaceable arrangement of first and second housing components may be designed in a variety of different ways. For instance, first and second housing components can be slidably disposed relative to each other. They might be displaceable relative to each other in a nested or telescope-like configuration. Also, a threaded or toothed engagement, also a positive engagement of first and second housing components is conceivable.

The relative displacement of first and second housing components may not only serve to eliminate axial clearance between piston rod and piston. It may also be applicable to eliminate backslash and clearance of drive mechanism itself. By means of the clearance eliminating motion, also the piston rod itself might become subject to axial thrust, effectively eliminating clearance or backslash being inherent of the drive mechanism.

Since the first housing component is adapted to receive the cartridge comprising the medicinal product and the second housing component is adapted to essentially house the dose dispensing mechanism, the first housing component acts as a cartridge holder. It may therefore comprise a receptacle featuring a shape and geometry according to the cartridge's contour. Preferably, the cartridge is retained in said first housing component in an immobile way. Since the second housing component retains the dose dispensing mechanism and its piston rod, by way of mutually displacing first and second housing components in axial direction, the distance between the cartridge's piston and the piston rod varies correspondingly.

In particular with non-reusable but disposable drug delivery devices in a pre-assembly configuration, the cartridge is retained in the first housing component and the dose dispensing mechanism and its piston rod is retained in the second housing component. In a final assembly step, i.e. when first and second housing components are mechanically coupled and connected, first and second housing components can be directly arranged in such a manner, that the piston rod's distal end and the piston's proximal end face abut and get in mechanical contact. In this way, clearance-elimination can be directly embedded in the assembly procedure of the drug delivery device and a separate clearance-elimination step, in particular, a user-governed set-up procedure is no longer required.

According to a further preferred aspect, the interlock means is adapted to mutually lock first and second housing components irrespective of their relative position. In this way, first and second housing components can be mutually locked in place, preferably when axial clearance of the drug delivery device has been eliminated. In this context, it is conceivable, that in a final assembly configuration, first and second housing components are arranged in an at least partially nested manner. First and second housing components at least partially overlap. It is also conceivable, that in said final assembly configuration, first and second housing components remain axially separated. However, by way of the interlock means, their relative position can be fixed, such that the clearance-eliminated configuration of the drug delivery device remains unchanged.

According to another embodiment, in an adjusting mode, first and second housing components are mechanically disengaged and free to be moved in axial direction. In said adjusting mode, the interlock means is at least temporally disabled, such as to allow for the relative movement of first and second housing components. Particularly, a smooth axial displacement of first and second housing components is provided, allowing for an intuitive elimination of clearance between piston and piston rod. As soon as piston and piston rod mutually abut, a haptic feedback is perceptible indicating the elimination of clearance between piston and piston rod.

According to a further preferred embodiment, the interlock means is designed as positive, frictional or adhesive lock. The interlock means may further be of reversible type, such as to allow a disassembly of the drug delivery device, e.g. for replacing of a used cartridge. Then, the interlock means is designed as a releasable and reversible interlock feature. Alternatively, the interlock means may also be designed as irreversible interlock, which is particularly applicable for disposable and non-reusable drug delivery devices. By activating the interlock means, the drug delivery device can be switched into a locking mode, in which relative displacement of first and second housing components is impeded.

In another embodiment, the interlock means is adapted to provide a retention force being substantially larger than an axially directed force required to displace or to move first and second housing components relative to each other. In particular, the retention force to be provided by the interlock means is substantially larger than the sum of static and dynamic friction forces required to axially move first and second housing components relative to each other. Moreover, the retention force also exceeds axial forces arising under normal operating conditions, i.e. when axial thrust is transferred from the piston rod to the cartridge's piston. Furthermore, the interlock means and its interaction with at least one of the housing components has to be designed and dimensioned in such a way, that the mutual locking into position of first and second housing components remains unaffected during and after repeated sequences of dose setting and dose dispensing procedures.

In a further preferred embodiment, the first or the second housing component comprises a receptacle to slidably receive at least a neck portion of the other, second or first housing component. In this way, first and second housing components are arranged in a nested or telescope-like way, allowing for an adjustment in length of the entire housing. In another embodiment, mutually corresponding receptacle and neck portion of first and second housing components may comprise respective means to inhibit a rotation of first and second housing components relative to each other. The stepped down neck portion at its outer circumference and the receptacle at its inside facing wall may therefore comprise mutually corresponding rotation-inhibiting means, such as splines and corresponding grooves.

Alternatively, instead of a purely axial sliding displacement, it is also conceivable, that first and second housing components are threadedly engaged in such a way, that their relative axial position or distance varies due to a relative rotation of first and second housing components.

According to another preferred embodiment, the interlock means comprises a locking ring, which is displaceably arranged in axial direction on the outer circumference of the first and/or the second housing component. Preferably, the locking ring is axially displaceably disposed on the outer circumference of that particular housing component comprising the receptacle adapted to receive the corresponding stepped down neck portion of the other housing component. The locking ring might be slideably disposed on said housing component in axial direction. Alternatively or additionally, it is also conceivable, that the locking ring and said housing component are threadedly engaged, such that the locking ring may act as a locking nut.

Preferably, the locking ring is adapted to exert a radially inwardly directed force to the outer circumference of the respective housing component. Alternatively, the locking ring may be adapted to impede a radial widening of the respective housing component. Since the locking ring is typically disposed in an overlapping region of first and second housing components, axial displacement of said locking ring may lead to an intended positive and/or frictional engagement of receptacle and neck portion of first and second housing components. Furthermore, the locking ring may comprise a somewhat conical structure such as to exert a radially inwardly directed force effect on the outer circumference of first and/or second housing component when displaced in axial direction.

According to another preferred embodiment, receptacle and neck portion of said first and housing components comprise mutually corresponding toothed surfaces. The toothed surfaces may comprise a micro-detent structure, which may already provide a kind of self-locking effect. Still, these toothed surfaces are adapted to provide a smooth-running relative displacement of first and second housing components for the purpose of clearance elimination. The tooth-geometry may be symmetric or asymmetric. In an asymmetric embodiment, the toothed surfaces on the one hand may allow for a relative smooth and smooth-running axial displacement of first and second housing components towards each other. On the other hand, the particular design of the toothed surfaces may impede or at least provide enlarged resistance against a movement of first and second housing components in opposite directions.

According to another aspect of the invention, the interlock means comprises bonding or welding of radially overlapping receptacle and neck portion of said first and second housing components. This way, a non-reversible coupling and connection of first and second housing components is generated. Bonding can be performed by supplying an appropriate adhesive, preferably, before first and second housing components are assembled. For instance, corresponding neck portion and receptacle of first and second housing components can be coated or provided with separate components of a two-component adhesive. When reaching the final assembly position, the adhesive can be cured by supplying appropriate thermal energy.

The first and second housing components can also be fixed or immobilized by an at least punctual welding in their overlapping region. Welding of first and second housing components can be easily conducted, typically by means of laser radiation, wherein the receptacle at least comprises portions being at least partially transparent for radiation to be used. In this way the housing components can be fused and joined by casting.

According to another preferred embodiment, the first and/or second housing components are oval or elliptic in diameter, at least in a section where first and second housing component overlap in radial direction. In particular, the first housing component is at least partially insertable into the corresponding second housing component. Hence, by rotating the oval or elliptic housing component with respect to its cylinder-like long axis, a radial clamping can be achieved, at least allowing for a respective pre-fixing of first and second housing components. Thereafter, first and second housing components may also become subject to a welding or gluing process, e.g. for immobilizing the two housing components permanently. Alternatively, also the first housing component may comprise a receptacle adapted to receive the oval or elliptically shaped second housing component.

Preferably, both housing components at least partially comprise an oval or elliptic cross sectional area, wherein for instance one housing component comprises a receptacle being oval or elliptic in diameter and wherein the other housing component comprises a corresponding oval or elliptically shaped cross section allowing for a mutual at least partial insertion of said housing components. If the two oval shaped housing components are oriented with their major and/or minor axis in parallel, a radial gap will form between first and second housing components allowing for a smooth assembly of the two housing components. In the course of clearance elimination, as soon as an abutment position of piston and piston rod has been reached, the two housing components can be mutually braced., e.g. by twisting or rotating the two components relative to each other.

With these embodiments, the interlock means are represented by the shape and/or geometry of first and/or second housing components. Hence, the interlock means comprise an oval shaped cross section of first and/or second housing components, and in particular an oval shaped receptacle or neck portion.

In another independent aspect, the invention relates to a method of eliminating clearance between a piston and a piston rod of a drug delivery device. The drug delivery device comprises a first housing component, which is adapted to receive a cartridge. The cartridge itself comprises a medicinal product and further comprises a piston displaceably arranged in an axial direction for dispensing of a dose of the medicinal product. For the purpose of dose dispensing, the piston is operably engaged with a piston rod of a dose dispensing- or drive mechanism of the drug delivery device which is to be arranged in a second housing component. The method of eliminating clearance between piston and piston rod during or after assembly of the device comprises the steps of moving the first housing component in axial direction relative to the second housing component until axial clearance is eliminated. Thereafter, first and second housing components are mutually locked into position by means of an interlock means. In this way axial clearance between the cartridge's piston and the piston rod but also axial clearance and backslash of the dose dispensing mechanism itself can be eliminated or at least reduced to an acceptable minimum.

The method is particularly applicable in a mass production or mass manufacturing process, wherein the first and second housing components are preassembled with the cartridge and the drive mechanism, respectively.

The present method for clearance- or backslash elimination characterises in a two step process, wherein in an adjusting mode first and second housing components can be moved relatively easily and smooth in axial direction with respect to each other. In this way, abutment of the piston rod's distal portion and the piston's proximal end face can be easily and unambiguously sensed and detected. In a subsequent step, the device is switched into an interlocking mode by activating the interlock means. In the interlock mode, relative axial displacement of first and second housing components is substantially impeded.

In a preferred embodiment, prior to assembly of the device and prior to assembly of first and second housing components, the cartridge is arranged in or at the first housing component. Furthermore, also the dose dispensing mechanism and its piston rod is arranged in or at the second housing component. Cartridge and dose dispensing mechanism are typically fixed to respective first and second housing components. By way of an axial relative displacement of first and second housing components, also the piston rod and the cartridge's piston become subject to a respective, hence clearance eliminating relative displacement.

In a further preferred embodiment, the interlock means comprises a locking ring displaceably arranged in axial direction on the outer circumference of first and/or second housing components. Typically, the locking ring is arranged in an overlapping, nested or interleaved region of first and second housing components, so as to provide a radially inwardly acting retention force, when the device is in its clearance-free configuration.

In another alternative or supplemental embodiment, radially overlapping sections of first and second housing components, e.g. radially overlapping neck portion and corresponding receptacle of first and second housing components are mutually bonded or welded for locking into position first and second housing components, when a final assembly configuration, in which clearance has been substantially eliminated, has been reached. In embodiments, wherein at least one of first or second housing components comprises an oval or elliptical cross section, a clamping by rotation of first and second housing components can be established.

The term “medicament” or “medicinal product”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, a antibody, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art, that various modifications and variations can be made to the present invention without departing from the spirit and scope of the invention. Further, it is to be noted, that any reference signed used in the appended claims are not to be construed as limiting the scope of the present invention.

Without limitation, the present invention will be explained in greater detail below in connection with preferred embodiments and with reference to the drawings in which:

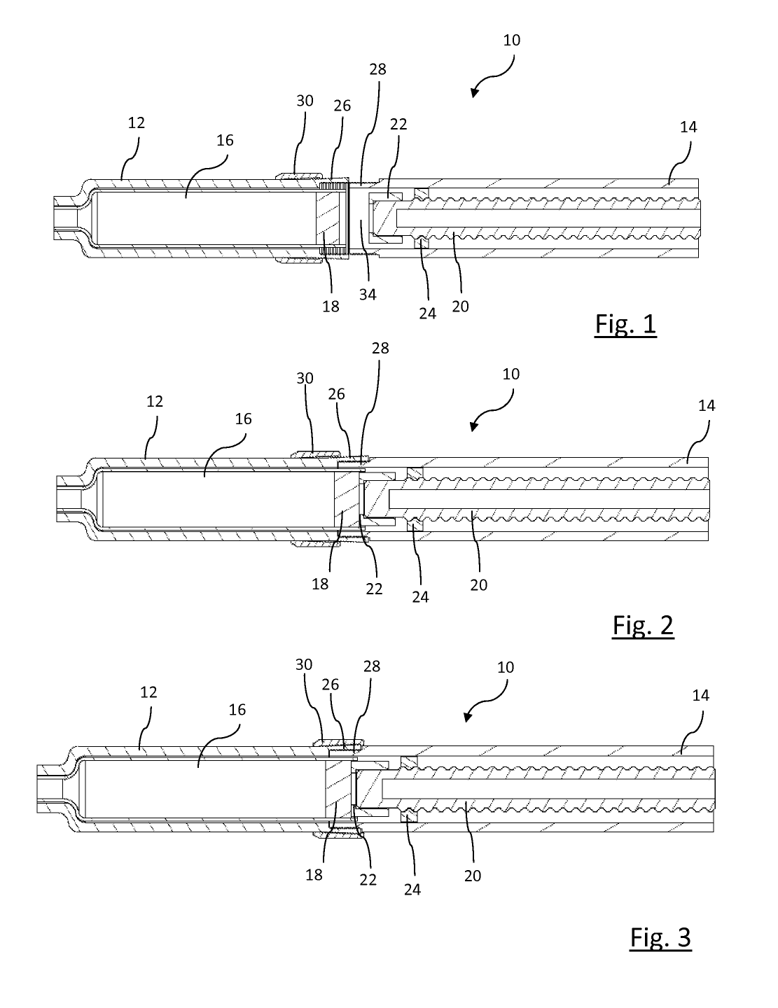

FIG. 1 schematically illustrates a first embodiment of the invention in cross-sectional view in pre-assembly configuration,

FIG. 2 illustrates the embodiment according to FIG. 1 in its clearance-free final assembly configuration,

FIG. 3 depicts the device according to FIGS. 1 and 2 in interlock mode,

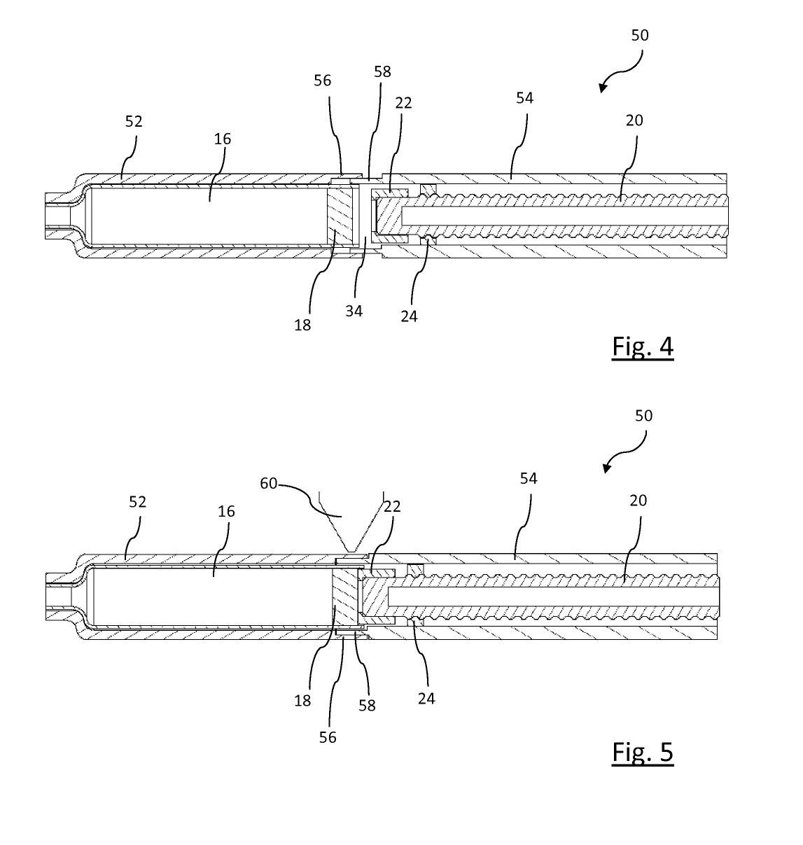

FIG. 4 schematically illustrates in cross-sectional view of a second embodiment of the present invention in pre-assembly configuration,

FIG. 5 shows the embodiment according to FIG. 4 in a final assembly configuration,

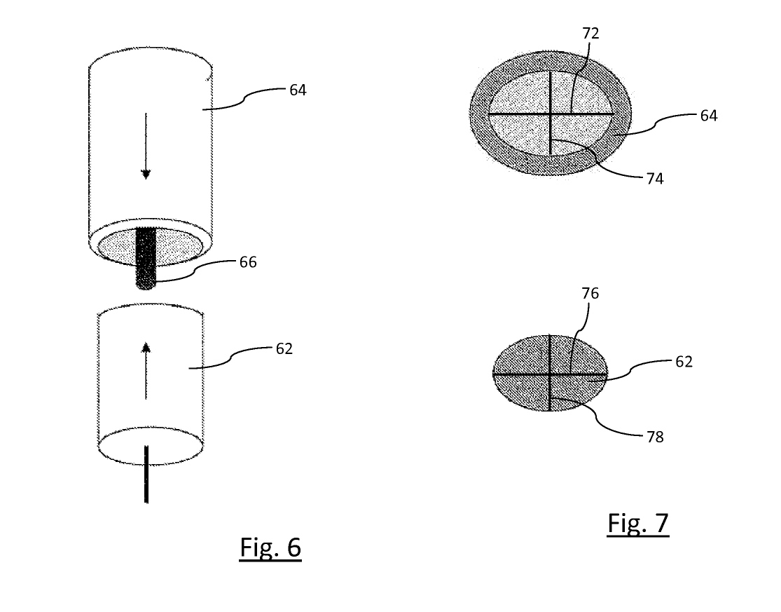

FIG. 6 schematically illustrates another embodiment of the invention, wherein the housing components comprise an oval or elliptic shape,

FIG. 7 separately shows the two housing components in diameter,

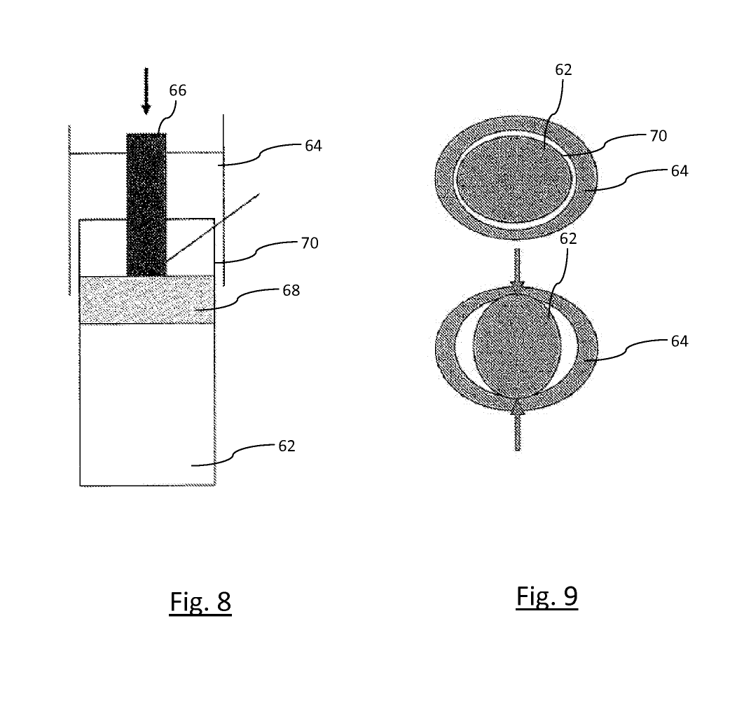

FIG. 8 depicts a longitudinal cross sectional illustration of first and second housing component in an assembly configuration and

FIG. 9 shows the two housing components in unlocked and locked configuration.

In FIGS. 1 to 3, a drug delivery device 10 according to a first embodiment is exemplary illustrated. The drug delivery device 10 designed a s pen-type injector has a two-component housing 12, 14, wherein the distal housing component 12 serves as cartridge holder for a cartridge 16 filled with a medicinal product to be dose-wise dispensed. At its distal section, which is illustrated on the left hand side in FIGS. 1 to 3, the cartridge holder 12 comprises a stepped down neck portion, serving as a receptacle for the cartridge 16 retained therein.

In proximal direction, directed to the right hand side in FIGS. 1 to 3, the cartridge 16 comprises an axially slidably arranged piston 18, which—under an impact of a distal movement of a driven piston rod 20—is stepwise moved in distal direction for the purpose of expelling or purging a predefined amount of the medicinal product contained in the cartridge 16. The piston rod 20 is radially secured by means of a mount 24. The mount 24 is typically provided with a threaded circular opening extending thereto. It may also be integrally formed with the housing component 14 in the form of a radially inwardly directed flange having an internal thread. The piston rod 20 is axially displaceable by means of a not further illustrated dose dispensing mechanism or drive mechanism.

The cartridge holder 12 is to be coupled or connected with a proximal, second housing component 14, which retains the axially displaceable piston rod 20. Cartridge holder 12 and proximal housing component 14 are arranged in an interleaved or nested manner. A stepped down neck portion 28 of the proximal, second housing component 14 is received in a corresponding receptacle 26 of a proximally located receiving portion of the first housing component 12, that serves as cartridge holder.

In FIG. 1, there is further illustrated a gap 34 between a distal end section 22 of the piston rod 20 and a proximal end face of the piston 18. The axial size of this clearance or gap 34 may vary due to manufacturing and geometric tolerances of the components of the drug delivery device 10 or due to varying positions of the piston 18 with respect to the cartridge 16. In order to eliminate the gap 34, first and second housing components 12, 14 are displaceably arranged in axial direction relative to each other. Hence, the axial extension of mutually corresponding neck portion 28 and receptacle 26 determine an adjustment path for the relative displacement of first and second housing components 12, 14. The axial extension of the adjustment path provided by receptacle 26 and stepped down neck portion 28 typically exceeds the maximal axial size of the gap 34.

In the pre-assembly configuration as illustrated in FIG. 1, the cartridge 16 is pre-assembled in the cartridge holder 12 and the piston rod 20 together with its distally arranged thrust piece 22 is pre-assembled in the proximal housing component. In order to eliminate axial clearance between piston 18 and piston rod 20, 22, the two housing components 12, 14 are displaced in axial direction relative to each other, as becomes apparent by a comparison of FIG. 1 and FIG. 2. In the final assembly configuration as depicted in FIG. 2, the gap 34 has been eliminated. The thrust piece 22 is disposed to abut the proximal end face of the piston 18. Additionally, the receptacle 26 disposed at the proximal end of the cartridge holder 12 now almost entirely receives the stepped down neck portion 28 of the proximal housing component 14.

The ways on how to axially displace first and second housing components 12, 14 relative to each other are manifold. For instance, the outer surface of the stepped down neck portion 28 and the corresponding inner surface of the receptacle's 26 side wall may comprise a clogged surface allowing for a smooth-running axial displacement of first and second housing components 12, 14. Alternatively, corresponding surfaces of neck portion 28 and receptacle 26 may comprise toothed surfaces providing a particular self-locking function by virtue.

However, such toothed surfaces should still provide at least a smooth-running unidirectional adjustment of first and second housing components 12, 14.

In a further alternative embodiment, it is even conceivable, that receptacle 26 and neck portion 28 are threadedly engaged. In such a configuration, clearance 34 can be eliminated by a relative rotational movement of first and second housing components 12, 14 with their cylinder long axis as axis of rotation. In this context it is even conceivable to at least partially implement the interlock means into receptacle 26 and neck portion 28 themselves. For instance, when the threaded engagement is of self-locking type, the interlock means only have to secure first and second housing components 12, 14, against unintentional relative rotation when clearance elimination as depicted in FIG. 2 has been reached.

In FIG. 3, an interlock mode of the drug delivery device 10 is exemplary illustrated. Here, the interlock means is designed as circumferential ring 40, that circumferentially extends about the proximal portion of the cartridge holder 12. The securing and interlocking of the two housing components 12, 14 becomes apparent by a comparison of FIG. 3 and FIG. 2. By shifting the locking ring 30 in proximal direction, into the region where receptacle 26 and neck portion 28 radially overlap, the locking ring 30 impedes a radial widening of the receptacle 26. Additionally or alternatively, the ring 30 may also serve to exert a radially inwardly directed retaining force to the receptacle 26. In this way, receptacle 26 and neck portion 28 may become frictionally engaged.

In particular, when receptacle 26 and neck portion 28 are provided with mutually corresponding toothed surfaces, the locking ring might be adapted to prevent a radial widening of the receptacle 26. In this way, mutually engaged toothed or geared surfaces of receptacle 26 and neck portion 28 remain engaged and a relative axial motion of the two housing components 12, 14 is effectively prevented.

The locking ring 30 may be slideably disposed at the outer circumference of the cartridge holders 12 in proximal direction. Preferably, the locking ring 30 itself can be locked against axial displacement, in particular, when the locking ring 30 is in its proximal end position as depicted in FIG. 3. Alternatively, also a threaded engagement of locking ring 30 and cartridge holder 12 is conceivable. Hence, the locking ring 30 may be designed as locking nut comprising an internal thread engaged with a circumferential thread of the cartridge holder 12.

The embodiment according to FIGS. 4 and 5 shows a different solution for an interlock means. Also here, the drug delivery device 50 comprises a cartridge holder 52 and a proximal housing component 54. In a pre-assembly configuration as depicted in FIG. 4, the piston rod 20 with its distally disposed thrust piece 22 is pre-assembled in the housing component 54. It is radially secured by means of the mount 24 and it is axially driven by means of a not further illustrated drive mechanism.

The distally located cartridge holder 52 at its proximal end section comprises a receptacle 56, adapted to receive a corresponding stepped down neck portion 58 of the proximal housing component 54. In the pre-assembly configuration of FIG. 4, an axial gap 34 exists between a proximal end face of the piston 18 and a distal end face of the thrust piece 22. In order to annihilate the gap 34 and for eliminating axial clearance or backslash, the two housing components 52, 54 are axially disposed relative to each other until abutment of thrust piece 22 and piston 18 has been reached, as illustrated in FIG. 5.

Mutually corresponding neck portion 58 and receptacle 56 comprise rather clogged surfaces allowing for a smooth-running relative axial displacement. As soon as the clearance-eliminated configuration as depicted in FIG. 5 has been reached, first and second housing components 52, 54 have to be mutually interlocked. In FIG. 5, a laser or welding apparatus 60 is schematically illustrated for supplying thermal heat to the overlapping region formed by receptacle 56 and neck portion 58. In this way receptacle 56 and/or stepped down neck portion 58 can be partially melt on, so that the two housing components 52, 54 become mutually fixed and joined.

Preferably, when making use of a laser welding process, it is beneficial, when the side wall of the receptacle 56 comprises surface portions being at least partially transparent for the respective laser radiation. Preferably, the neck portion 58 of the second housing component 54 is highly absorbent for the laser radiation of choice. This way, a melt on and bonding of the two housing components 52, 54 can be generated in the mutually intersecting or overlapping region of the two housing components.

Alternatively or additionally, it is conceivable to supply thermal energy to the overlapping portion of first and second housing components 52, 54, leading in effect to an at least partial melt of the radially outwardly disposed housing component for the purpose of bonding first and second housing components.

In FIGS. 6 through 9 another embodiment of the present invention is illustrated, wherein the two housing components 62, 64 are at least partially of oval or elliptic cross section. In the upper sketch of FIG. 7, the second housing component 64 is shown in cross section, whereas a corresponding cross section of the first housing component 62 is illustrated in the lower sketch. As depicted, the second housing component 64 is slightly larger in diameter and in cross section than the first housing component 62. Therefore, the first housing component 62 can be displaced in proximal direction, hence upwards, towards the second housing component 64, which serves as a receptacle to receive a proximal end section of the first housing component 62.

The major and minor axes 74, 76 of the receptacle of the second housing component 64 are larger than respective major and minor axes 76, 78 of the first housing component 62. In this way, when first and second housing components 62, 64 are oriented in such a way, that their respective major and minor axis 72, 74, 76, 78 are substantially in parallel, the first housing component 62 can be introduced into the second housing component's receptacle.

Preferably, the outer diameter of the first housing component 62 is substantially smaller than the inner diameter of the second housing component 64. In this way, a radial gap 70, as shown in the upper sketch of FIG. 8 establishes, allowing for a smooth insertion of the first housing component 62 into the second housing component 64.

When axial clearance, preferably between piston 68 and piston rod 66 has been eliminated, the two housing components 62, 64 can be mutually locked in position, e.g. by twisting or rotating first and/or second housing components 62, 64 with respect to each other. In this way, a mutual interlocking or at least an axial pre-fixing of first and second housing components 62, 64 can be achieved by way of a radial clamping, as illustrated in the lower sketch of FIG. 9.

It is further to be noted, that for the embodiment according to FIGS. 6 to 9, generally, it is only required, that the outer shape of the first housing component 62 comprises a non-circular, oval or elliptic cross section. The receptacle or receiving portion provided by the second housing component 64 can be of e.g. circular shape. Independent on whether the receptacle of the second housing component 64 is of circular or oval shape, its outer surface can be designed arbitrarily.

### LIST OF REFERENCE NUMERALS

10 drug delivery device
12 housing component
14 housing component
16 cartridge
18 piston
20 piston rod
22 thrust piece
24 mount
26 receptacle
28 neck portion
30 locking ring
34 clearance
50 drug delivery device
52 housing component
54 housing component
56 receptacle
58 neck portion
60 laser/welding apparatus
62 cartridge holder
64 housing
66 piston rod
68 piston
70 radial gap
72 major axis
74 minor axis
76 major axis
78 minor axis

1. Drug delivery device for dispensing of a dose of a medicinal product, comprising:
a first housing component adapted to receive a cartridge comprising the medicinal product and further comprising a piston slidably arranged therein in an axial direction,
a second housing component adapted to receive a dose dispensing mechanism having a piston rod to be operably engaged with the cartridge's piston,
wherein the first and the second housing components are displaceably arranged in axial direction relative to each other for reducing an axial distance between the piston and the piston rod to a pre-defined degree, and
an interlock means adapted to interact with at least one of first and second housing components for mutually locking into position first and second housing components.

2. The drug delivery device according to claim 1, wherein the interlock means is adapted to mutually interlock first and second housing components in an at least partially nested or interleaved configuration irrespective of their position relative to each other.

3. The drug delivery device according to claim 1, wherein the interlock means is transformable into a release mode in which first and second housing components are mechanically disengaged and are free to be moved in axial direction relative to each other.

4. The drug delivery device according to claim 1, wherein the interlock means designed as positive, frictional or adhesive interlock comprises an interlock member slidably and/or rotatably arranged with respect to the first and/or second housing component.

5. The drug delivery device according to claim 1, wherein the interlock member comprises a locking ring displaceably and/or slidably arranged in axial direction on the outer circumference of the first and/or second housing component.

6. The drug delivery device according to claim 1 wherein the first or second housing component comprises a receptacle to slidably receive at least a neck portion of the other, second or first housing component.

7. The drug delivery device according to claim 5, wherein the receptacle and the neck portion of said first and second housing components comprise mutually corresponding toothed surfaces.

8. The drug delivery device according to claim 6, wherein the interlock means comprises bonding or welding of radially overlapping receptacle and neck portion of said first and second housing components.

9. The drug delivery device according to claim 1, wherein the first and/or second housing component at least partially comprises an oval or elliptic cross section, wherein the first and second housing components are mutually lockable in position by rotating first and second housing components relative to each other.

10. A method of eliminating clearance between a piston and a piston rod of a drug delivery device comprising a first housing component and a second housing component, wherein the first housing component is adapted to receive a cartridge comprising a medicinal product and a piston displaceably arranged therein in an axial direction, and wherein the second housing component is adapted to house a drive mechanism comprising a piston rod to be operable engaged with the piston, the method of eliminating said clearance during or after assembly of the device comprises the steps of:
moving a first housing component in axial direction relative to a second housing component for reducing an axial distance between the piston and the piston rod to a pre-defined degree,
mutually locking into position first and second housing components by means of an interlock means.

11. The method according to claim 10, wherein prior to assembly of the device, the cartridge is arranged in or at the first housing component and the dose dispensing mechanism is arranged in the second housing component.

12. The method according to claim 10, wherein a locking ring displaceably arranged in axial direction on the outer circumference of the first and/or second housing component is axially shifted towards a proximal end section of the first housing component or towards a distal end section of the second housing component.

13. The method according to claim 10, wherein radially overlapping sections of first and second housing components are mutually clamped by rotation, bonded or welded for locking into position first and second housing components.
